(19) United States
(12) Patent Application Publication
(10) Pub. No.: US 2025/0381392 A1
(43) Pub. Date: Dec. 18, 2025

(54) Methods And Apparatuses For Inhibiting Movement Of Implanted Magnetic Devices

(71) Applicant: Cochlear Limited, Macquarie University, NSW (AU)

(72) Inventors: Jan Patrick Frieding, Grose Vale (AU); James Sier, Perth (AU); Caitlin Revell, Perth (AU); Emily Parkinson, Perth (AU); Sam Croston, Perth (AU); Jack Ellery, Perth (AU)

(73) Assignee: Cochlear Limited, Macquarie University, NSW (AU)

(21) Appl. No.: 18/876,463

(22) PCT Filed: Jun. 27, 2023
(86) PCT No.: PCT/IB2023/056591

§ 371 Date: Dec. 18, 2024

Related U.S. Application Data
(60) Provisional application No. 63/358,004, filed on Jul. 1, 2022.

(51) Int. Cl. A61N 1/08; A61N 1/36

(52) U.S. Cl. CPC A61N 1/08; A61N 1/36038; A61N 1/36046

(57) ABSTRACT

An apparatus is provided that inhibits movement of a magnetic device relative to a recipient in which the magnetic device is implanted that is caused by exposing the magnetic device to a magnetic field generated externally to the recipient. The apparatus includes a pressure applicator portion configured to apply pressure against the recipient at a location over the magnetic device when the apparatus is pressed against the recipient. The apparatus further includes a support portion configured to support the apparatus against the recipient at a different location than over the magnetic device when the pressure applicator portion applies pressure at the location over the magnetic device. The location and the different location are spaced apart from each other.

### CROSS REFERENCE TO RELATED APPLICATION

This patent application claims priority to U.S. provisional patent application 63/358,004, filed Jul. 1, 2022, which is incorporated by reference herein in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### BRIEF SUMMARY

According to a first aspect of the present invention there is provided an apparatus for inhibiting movement of a magnetic device relative to a recipient that is caused by exposing the magnetic device to a magnetic field generated externally to the recipient, wherein the magnetic device is implanted in the recipient. The apparatus comprises a pressure applicator portion configured to apply pressure against the recipient at a location over the magnetic device when the apparatus is pressed against the recipient, and a support portion configured to support the apparatus against the recipient at a different location than the location over the magnetic device when the pressure applicator portion applies pressure at the location over the magnetic device, wherein the location and the different location are spaced apart from each other.

According to a second aspect of the present invention there is provided a method for inhibiting motion of an implanted magnetic device in a recipient in response to a magnetic field that is generated externally to the recipient. The method comprises placing an apparatus on the recipient such that a first portion of the apparatus contacts the recipient at a location that is over the implanted magnetic device, and such that a second portion of the apparatus contacts the recipient at a location adjacent to the implanted magnetic device. The method further comprises securing the placed apparatus to the recipient such that the first portion and the second portion are pressed against the recipient so as to inhibit movement of the magnetic device while the magnetic device is exposed to the magnetic field.

According to a third aspect of the present invention there is provided a device for limiting motion of a magnetic implant in a recipient caused by an externally generated magnetic field. The device comprises a first portion that is able to apply pressure on the magnetic implant when the device is secured to the recipient, and a second portion that is able to limit movement of the secured device relative to the recipient during exposure of the magnetic implant to the externally generated magnetic field.

### BRIEF DESCRIPTION OF THE DRAWINGS

FIG. 1 depicts an exemplary embodiment of a neural prosthesis in general, and a retinal prosthesis and an environment of use thereof, in particular.

FIG. 2A is a diagram that depicts an example of an external component of a cochlear implant system.

FIG. 2B is a diagram that depicts a front view of an example of an implant of a cochlear implant system.

FIG. 2C is a diagram that depicts portions of a cochlear implant system including the external component of FIG. 2A attached magnetically to the implant of FIG. 2B that has been implanted in a recipient.

FIG. 3 is a diagram that depicts a side view of the implant shown in FIG. 2B for the cochlear implant system, and how a magnetic device of the implant, which is in the form of a retention magnet, can move relative to the rest of the implant.

FIG. 4 is a diagram that depicts an example of an apparatus that is configured to be placed over a magnetic device implanted in a recipient to inhibit movement of the magnetic device during exposure of the magnetic device to an externally generated magnetic field.

FIGS. 5A-5B are diagrams that depict an example of the apparatus of FIG. 4 placed on the head of a cochlear implant recipient.

FIGS. 6A-6B are diagrams that depict an example of a securing mechanism in the form of a strap that is configured to be wrapped around the head of a recipient over the apparatus of FIG. 4 in order to secure the apparatus to the head of the recipient and thereby reduce movement of the apparatus relative to the head of the recipient.

FIGS. 7A-7B are diagrams that depict another example of an apparatus that is configured to be placed at a location over a magnetic device implanted in a recipient to inhibit motion of the magnetic device during exposure of the magnetic device to a magnetic field generated externally to the recipient.

FIGS. 8A-8B are diagrams that depict an example of an implant locator for use with the apparatus depicted in FIGS. 7A-7B.

FIG. 9 is a diagram that depicts additional examples of apparatuses that are each configured to be placed over a magnetic device implanted in a recipient so as to inhibit motion of the magnetic device during exposure to an externally generated magnetic field.

### DETAILED DESCRIPTION

Merely for ease of description, the techniques presented herein are primarily described herein with reference to an illustrative medical device, namely a cochlear implant. However, it is to be appreciated that the techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant, corresponds to a disclosure of another embodiment of using such teaching with another hearing prostheses, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc.

The techniques presented herein can be used with implantable/implanted microphones, whether or not used as part of a hearing prosthesis (e.g., a body noise or other monitor, whether or not it is part of a hearing prosthesis). The techniques presented herein may be used with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc., and thus any disclosure herein is a disclosure of utilizing such devices with the teachings herein. At least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis, such as a retinal prosthesis. In this regard, any disclosure herein with respect to a hearing percept corresponds to a disclosure of other types of neural percepts in other embodiments, such as a visual/sight percept, a tactile percept, a smell precept or a taste percept, unless otherwise indicated. The teachings detailed herein can be implemented in any type of magnetic implants, such as magnets in hearing implants, cosmetic implants, eye implants, etc.

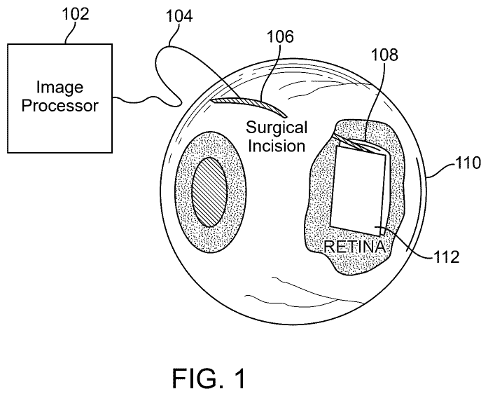

FIG. 1 presents an exemplary embodiment of a neural prosthesis in general, and a retinal prosthesis and an environment of use thereof, in particular. In some embodiments of a retinal prosthesis, a retinal prosthesis sensor-stimulator 108 is positioned proximate the retina 110. In an exemplary embodiment, photons entering the eye are absorbed by a microelectronic array of the sensor-stimulator 108 that is hybridized to a glass piece 112 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 102 is in signal communication with the sensor-stimulator 108 via cable 104 which extends through surgical incision 106 through the eye wall (although in other embodiments, the image processor 102 is in wireless communication with the sensor-stimulator 108). The image processor 102 processes the input into the sensor-stimulator 108, and provides control signals back to the sensor-stimulator 108 so the device can provide processed and output to the optic nerve. That said, in an alternate embodiment, the processing is executed by a component proximate to or integrated with the sensor-stimulator 108. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 108 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

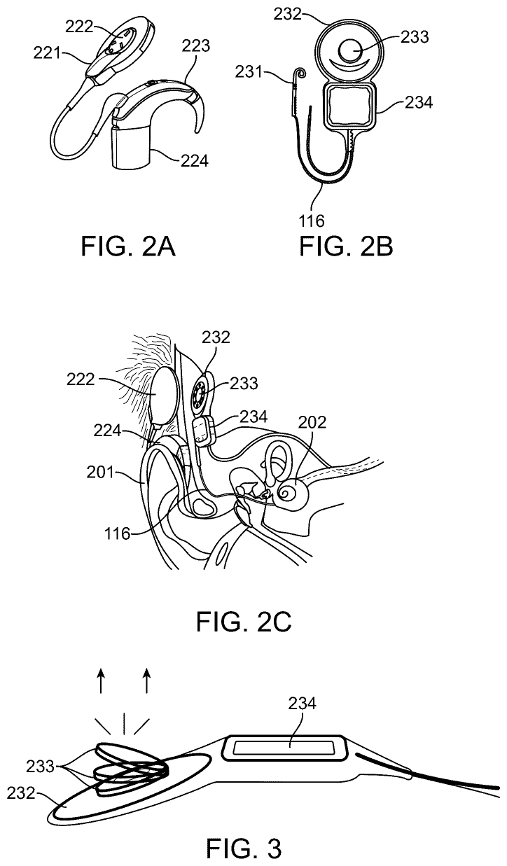

According to some exemplary embodiments disclosed herein, a pressure applicator apparatus can inhibit movement of a magnetic device implanted in a recipient in a cochlear implant system during exposure of the magnetic device to an externally generated magnetic field. Figures (FIGS. 2A-2C of the present disclosure relate to an exemplary cochlear implant system that can be used with techniques disclosed herein. Although, the teachings detailed herein are applicable to any type of magnetic device implanted in a recipient, including any type of implantable device that emits a magnetic field or is magnetic. The magnetic device can be implanted in any part of a recipient's body, for example, on a hard surface like a head or above another bone of the recipient's body. The magnetic device can be any size, have any shape, have any suitable strength, or have any suitable configuration or orientation with respect to the recipient.

FIG. 2A is a diagram that depicts an example of an external component for an exemplary cochlear implant system. FIG. 2B is a diagram that shows an example of an implant for the exemplary cochlear implant system. FIG. 2C is a diagram that shows portions of the exemplary cochlear implant system including the external component of FIG. 2A coupled magnetically to the implant of FIG. 2B that has been implanted in a recipient. For ease of illustration, FIGS. 2A-2C are described together herein.

The external component of the cochlear implant system shown in FIG. 2A is directly or indirectly attached to the body of a recipient and typically includes an external coil 221 and an external magnet 222 fixed relative to the external coil 221. The external component of FIG. 2A also includes one or more input devices 223 receiving input signals at a sound processing unit 224. In this example, the one or more input devices 223 include sound input devices (e.g., microphones positioned by an auricle of the recipient, telecoils, etc.) configured to capture/receive input signals. The sound processing unit 224 of FIG. 2A is a behind-the-ear (BTE) sound processing unit configured to be attached to, and worn adjacent to, a recipient's ear 201, as shown in FIG. 2C.

The implant for the cochlear implant system shown in FIG. 2B includes an intra-cochlear stimulating assembly 231, a pocket 232 containing an internal magnet 233, a receiver/stimulator unit 234, and lead region 116, all configured to be implanted under the skin/tissue of a recipient. The pocket 232, internal magnet 233, receiver/stimulator unit 234, and lead region 116 are shown in FIG. 2C implanted in a recipient. The pocket 232 may, for example, include a thin lip of silicone that is used to hold the internal magnet 233 and to allow for the magnet 233 to be more easily removed. The receiver/stimulator unit 234 generally includes a hermetically-sealed housing in which magnetic induction and/or radio frequency (RF) interface circuitry and a stimulator unit are disposed.

In the external component of FIG. 2A, the external coil 221 is fixed to the external magnet 222. In the implant of FIG. 2B, an implantable coil (not shown) is fixed to the internal magnet 233. The external and internal coils can be, for example, wire antenna coils each including multiple turns of electrically insulated single-strand or multi-strand wire. The magnets 222 and 233 facilitate the operational alignment of the external coil 221 with the implantable coil, and retention of the external coil 221 on the recipient's head. This operational alignment of the coils enables the external component of FIG. 2A to transmit data, as well as possibly power, to the implant of FIG. 2B via a closely-coupled wireless link formed between the external coil 221 with the implantable coil. In certain examples, the closely-coupled wireless link is a radio frequency (RF) link. However, various other types of energy transfer, such as infrared (IR), electromagnetic, capacitive and inductive transfer, can be used to transfer the power and/or data from the external component of FIG. 2A to the implant of FIG. 2B.

The stimulating assembly 231 is configured to be at least partially implanted in the recipient's cochlea 202. Stimulating assembly 231 includes a plurality of longitudinally spaced intra-cochlear electrical stimulating contacts (e.g., electrodes) that collectively form a contact or electrode array for delivery of electrical stimulation (e.g., current) to the recipient's cochlea. Stimulating assembly 231 extends through an opening in the recipient's cochlea (e.g., cochleostomy, the round window, etc.) and has a proximal end connected to the receiver/stimulator unit 234 via lead region 116 and a hermetic feedthrough (not shown in FIG. 2B). Lead region 116 includes a plurality of conductors (wires) that electrically couple the electrodes to the receiver/stimulator unit 234. A simulation unit in the receiver/stimulator unit 234 is configured to utilize stimulation control signals to generate electrical stimulation signals (e.g., current signals) for delivery to the recipient's cochlea via the one or more stimulating contacts in the stimulating assembly 231. The sound processing unit 224 generates the stimulation control signals by processing input audio signals. The input audio signals can be audio signals received via the sound input devices 223, signals received via auxiliary input devices in sound processing unit 224, and/or signals received via a wireless transceiver in sound processing unit 224. In this way, the cochlear implant system of FIGS. 2A-2C electrically stimulates the recipient's auditory nerve cells, bypassing absent or defective hair cells that normally transduce acoustic vibrations into neural activity, in a manner that causes the recipient to perceive one or more components of the input audio signals as sound.

Magnetic Resonance Imaging (MRI) is a medical procedure that often uses a powerful magnetic field and a computer to produce detailed images of internal portions of a human body. The images generated from an MRI can be used to help diagnose or monitor treatment for a variety of medical conditions. However, MRI and other medical procedures that generate magnetic fields are often inaccessible or difficult for people with cochlear implants and other types of magnetic implants, because the internal magnetic device implanted in the recipient can cause problems for implant recipients during these procedures. As an example, the internal magnet (e.g., internal magnet 233 shown in FIGS. 2B-2C) in a cochlear implant can vibrate or even dislocate during an MRI procedure, which can make the MRI procedure uncomfortable or even impossible for the recipient of the cochlear implant.

The internal magnet of a cochlear implant (such as internal magnet 233 shown in FIGS. 2B-2C) typically has low resistance to magnetic dislocation when a sufficient magnetic force is applied to the internal magnet. The internal magnet can consequently translate, rotate, and in some cases, flip within the recipient's head in response to the force applied by the changing magnetic field generated during an MRI procedure. A partially or fully dislocated internal magnet implanted in a recipient can cause concentrated pressures on the recipient's head, resulting in significant discomfort to the recipient and early termination of the MRI procedure. This outcome often leads to incomplete MRI scans, and sometimes revision surgery to relocate and/or replace the internal magnet, particularly in cases in which the internal magnet flipped within the recipient's head.

FIG. 3 is a diagram that depicts a side view of the implant shown in FIG. 2B for the cochlear implant system, and how the internal magnet 233 can move relative to the rest of the implant. FIG. 3 shows the internal magnet 233 dislocating in response to the magnetic field generated during an MRI procedure. The magnetic field generated during an MRI procedure may cause the internal magnet 233 to separate from the pocket 232 of the implant of the cochlear implant system, as shown for example in FIG. 3. The arrows in FIG. 3 indicate the direction of motion of the internal magnet 233 away from the pocket 232 in response to the magnetic field. The dislocation of the internal magnet 233 shown in FIG. 3 can cause significant discomfort for the recipient of the implant and early termination of the MRI procedure, as discussed above.

According to some preferred embodiments disclosed herein, an apparatus includes a pressure applicator portion configured to apply pressure against a recipient at a location over a magnetic device implanted in a recipient when the apparatus is pressed against the recipient in order to inhibit movement of the magnetic device when the magnetic device is exposed to a magnetic field generated externally to the recipient. The magnetic field can be generated during a procedure, such as an MRI or any other type of procedure that generates a magnetic field. The apparatus can also include a support portion configured to support the apparatus against the recipient at a different location than the location over the magnetic device when the pressure applicator portion applies pressure at the location over the magnetic device. The location and the different location are spaced apart from each other. The apparatus can also include a base portion from which both the pressure applicator portion and the support portion extend. The apparatus can be secured in place when the magnetic device is exposed to the magnetic field using a securing mechanism. The apparatus and the securing mechanism are placed on the recipient to apply enough force to the magnetic device to inhibit movement of the magnetic device during exposure to the magnetic field. By inhibiting movement of the magnetic device during exposure to the magnetic field, the comfort of the recipient is substantially improved, and the probability of being able to complete the procedure is substantially increased. The apparatuses and methods disclosed herein can be used to inhibit movement of a magnetic device that has been implanted internally within a recipient, such as a magnetic device in a cochlear implant, a magnetic device in a vestibular stimulator, a magnetic device in a visual implant, a magnetic device in a cosmetic implant, etc. The apparatuses and methods disclosed herein can be used to inhibit movement of a magnetic device implanted in any part of a recipient's body, such as the head, back, neck, arms, legs, or face of a recipient.

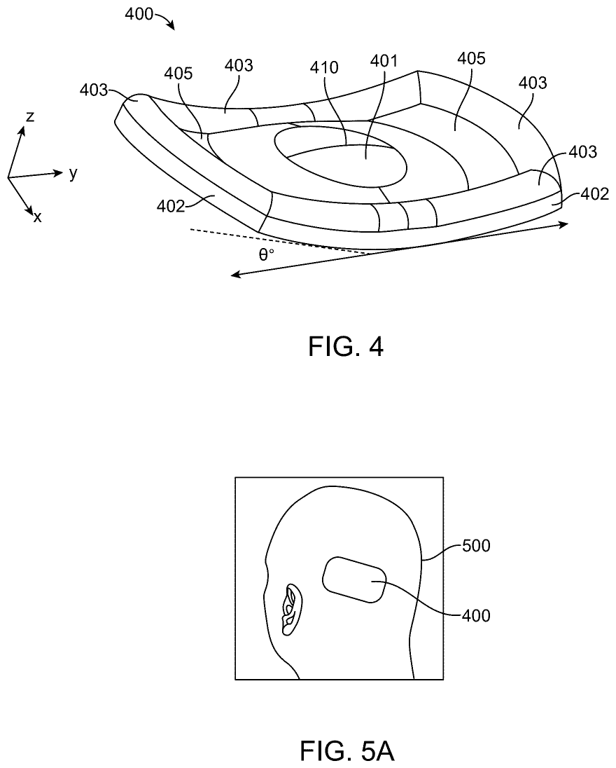

FIG. 4 is a diagram that depicts a preferred embodiment of an apparatus 400 that is configured to be placed over a magnetic device implanted in a recipient to inhibit movement of the magnetic device during exposure of the magnetic device to an externally generated magnetic field. Apparatus 400 may, for example, be a splint. Apparatus 400 includes a pressure applicator portion 401, a base portion 402, and a support portion 403. FIG. 4 also depicts a three dimensional x-y-z axis to assist in visualization of the apparatus 400. In the example of FIG. 4, the pressure applicator portion 401 of apparatus 400 is a pressure applicator mound that is mounted on the base portion 402. The base portion 402 has a surface 405 on one side of the apparatus 400. Surface 405 is exposed on both sides of the pressure applicator portion 401 along the length of apparatus 400 in the y direction. Surface 405 extends between, and is recessed relative to, the pressure applicator portion 401 and the support portion 403. The pressure applicator portion 401 of apparatus 400 extends in the z direction above the surface 405 of the base portion 402. The surface of the pressure applicator portion 401 curves up in the z direction to a central point 410 from surface 405 and from the sides of the pressure applicator portion 401 along the width of apparatus 400 in the x direction. In an alternative embodiment, the pressure applicator portion 401 can have, for example, a cylindrical shape (e.g., a pill shape), instead of a mound shape. In some embodiments, the pressure applicator portion 401 may be rotatable relative to the base portion 402.

The pressure applicator portion 401 is configured to apply a targeted pressure over the location of a magnetic device implanted in a recipient to inhibit movement of the magnetic device during exposure of the magnetic device to an externally generated magnetic field (e.g., during an MRI procedure). The pressure applicator portion 401 ensures that the pressure is localized over the magnetic device, reducing unnecessary pressure on other parts of the recipient's body. The pressure applicator portion 401 can also provide torque mitigation during exposure to an externally generated magnetic field by reducing rotational movement of the apparatus 400 against the recipient and increasing stability of the apparatus 400.

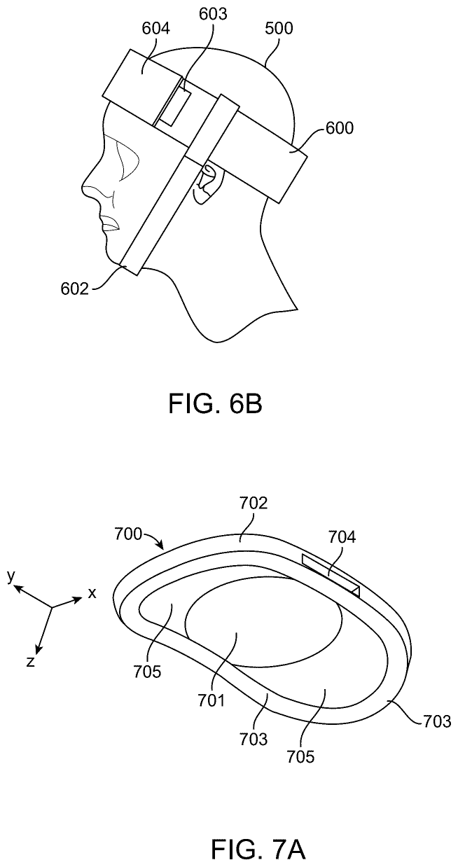

The base portion 402 of the apparatus 400 can be, for example, a backing plate made of a rigid material. In alternative embodiments, the base portion 402 can be made of a flexible or deformable material. In the example of FIG. 4, the base portion 402 has a curvature along its length in the y direction. The curvature of base portion 402 is concave relative to the surface 405 of apparatus 400. The curvature of the base portion 402 can be selected, for example, to fit the shape of the head (or other body part) of a typical recipient in likely implant locations to minimize protrusion of apparatus 400 from the recipient and to reduce movement of the apparatus 400 during exposure to an externally generated magnetic field. FIG. 4 illustrates that the base portion 402 of apparatus 400 has a curvature angle of θ°. θ can have any suitable value of 0° or greater. For example, θ can be 5°, 10°, 15°, 20°, 25°, etc. The curvature of the base portion 402 allows for an improved fit to the body of a recipient, helping to inhibit movement of the apparatus 400 during exposure to an externally generated magnetic field. The curvature of the base portion 402 also reduces the difficulty of sliding the apparatus 400 in below a strap around the recipient, as disclosed in further detail below with respect to FIGS. 6A-6B. In some embodiments, the base portion 402 can be flat (i.e., have a curvature θ of) 0°.

The support portion 403 is configured to support the apparatus 400 against the recipient at a different location than the location over the magnetic device when the pressure applicator portion 401 applies pressure at the location over the magnetic device. In the example of FIG. 4, the support portion 403 includes stabilizing bumpers mounted on the base portion 402. The support portion 403 extends above the surface 405 of the base portion 402 in the z direction around the perimeter of the base portion 402 on all 4 sides of the base portion 402. In other embodiments, the support portion may only be mounted on one, two, or three sides of the base portion. The support portion 403 can be higher than, or lower than, the highest point 410 of the pressure applicator portion 401 in the z direction. The support portion 403 helps to support the apparatus 400 against the recipient by applying pressure to additional locations on the recipient around the pressure applicator portion 401 to reduce lateral and rotational motion of the apparatus 400. The support portion 403 helps to ensure that the pressure applicator portion 401 maintains pressure over the location of the implanted magnetic device during exposure to an externally generated magnetic field. The support portion 403 also has a rounded surface to improve the comfort of the recipient and to reduce lateral and rotational motion of the apparatus 400.

The pressure applicator portion 401, the base portion 402, and the support portion 403 of the apparatus 400 can be made of any suitable non-magnetic materials. According to a specific example that is not intended to be limiting, any of the portions 401-403 of apparatus 400 can be made of a rigid, acrylonitrile butadiene styrene (ABS) polymer. ABS polymer has limited deformation, such that an apparatus 400 made with ABS polymer is sufficiently rigid to apply enough pressure over an implanted magnetic device to inhibit movement of the implanted magnetic device during exposure to an externally generated magnetic field. The ABS polymer can, in some examples, be manufactured using a three dimensional (3D) printing process. According to other specific examples that are not intended to be limiting, the bulk of one or more of the portions 401-403 of apparatus 400 can be made of any type of rigid or soft plastic.

According to additional examples that are not intended to be limiting, the surface of the pressure applicator portion 401 and/or the surface of the support portion 403 can, for example, be coated in a soft material, such as a food-grade (or medical grade) self-adhesive silicone-rubber sheet or lined with a thin foam lining. Coating or lining the surfaces of the pressure applicator portion 401 and/or the support portion 403 with a soft material can improve the comfort of the recipient during extended use of the apparatus 400. According to other examples, the pressure applicator portion 401 and/or the support portion 403 can be made of a flexible foam material that yields in response to the application of pressure to the apparatus 400 to improve the comfort of the recipient.

An apparatus that is used to inhibit motion of a magnetic device implanted in a recipient during exposure of the magnetic device to an externally generated magnetic field may be configured for a particular application or configured to fit any securing mechanism. According to various embodiments, an apparatus that is used to inhibit motion of a magnetic device implanted in a recipient during exposure of the magnetic device to an externally generated magnetic field can have any suitable dimensions or shape, such as a rectangular shape, a square shape, a circular shape, oval shape, or any other shape or size. According to a specific example that is not intended to be limiting, the base portion 402 of the apparatus 400 can be roughly 40 millimeters (mm) in width in the x direction and 60 mm in length in the y direction. According to still another example that is not intended to be limiting, the pressure applicator portion 401, the support portion 403, and the surface 405 can have smooth surface finishes that minimize bacterial collection and growth, increase presentability in a medical environment, and are washable.

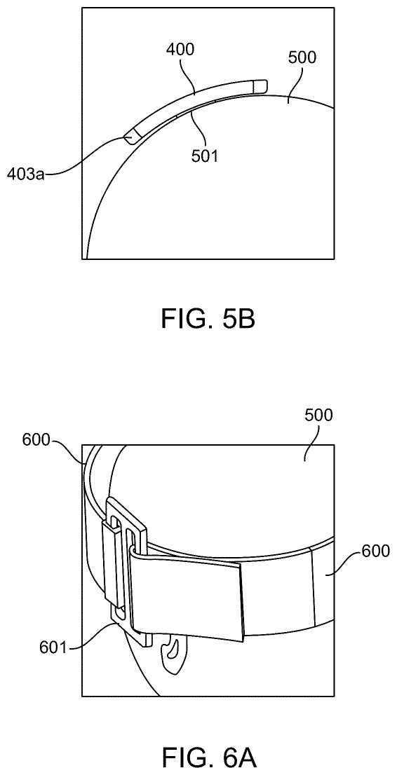

FIGS. 5A-5B are diagrams that illustrate different views of an example of the apparatus 400 of FIG. 4 placed on the head 500 of a cochlear implant recipient, according to an embodiment. In the example of FIGS. 5A-5B, a magnetic device is implanted under the skin of a recipient in the back of the head 500 at location 501 shown in FIG. 5B. The apparatus 400 is placed over the magnetic device such that the pressure applicator portion 401 is directly over the magnetic device in the recipient at location 501. The pressure applicator portion 401 is configured to apply targeted pressure to the magnetic device in the recipient in order to inhibit motion of the magnetic device during exposure of the magnetic device to an externally generated magnetic field. As an example, if the recipient has the implant of the cochlear implant system of FIG. 2B, the pressure applicator portion 401 can be placed directly over the internal magnet 233 at location 501. The apparatus 400 is placed on the recipient so that the apparatus 400 is closely aligned with the body of the recipient, as shown for example in FIG. 5B, prior to application of the externally generated magnetic field to the recipient. The support portion 403 of the apparatus 400 can, for example, contact all, or only a portion, of the head 500 of the recipient. In some applications, one or more parts of the support portion 403 may not be in direct contact with the head 500 of the recipient, such as region 403A shown in FIG. 5B. The parts of the support portion 403 that contact the head 500 of the recipient help to reduce movement of the apparatus 400 against the recipient.

FIGS. 6A-6B are diagrams that illustrate an example of a securing mechanism in the form of a strap that is configured to be wrapped around the head 500 of a recipient over the apparatus 400 in order to secure the apparatus to the head 500 of the recipient and thereby reduce movement of the apparatus 400 relative to the head 500 of the recipient. The securing mechanism of FIGS. 6A-6B is configured to be placed on the recipient so that the securing mechanism applies enough pressure to the apparatus 400 to hold the apparatus 400 on the recipient during exposure of the magnetic device to the externally generated magnetic field. The securing mechanism is configured to be secured to the recipient so that the securing mechanism is retained at the selected location on the body of the recipient during application of the externally generated magnetic field to the recipient.

The securing mechanism of FIGS. 6A-6B includes an adjustable strap/belt 600 that is configured to be placed over the apparatus 400 and wrapped around the head 500, as shown in FIGS. 6A-6B. The apparatus 400 can, for example, be placed at the back of the head 500 of the recipient as shown in FIG. 5A. The strap/belt 600 can be, for example, a thick compression bandage. The strap/belt 600 is looped through a buckle 601 to secure the strap/belt 600 to the head 500, as shown in FIG. 6A. The strap/belt 600 can be tightened or loosened by adjusting the length of the strap/belt 600 that is looped through the buckle 601. FIG. 6B shows a portion 604 of the strap/belt 600 that is looped through the buckle 601, folded back, and attached to the strap/belt 600 using a fastener 603 (e.g., Velcro) that is attached to the strap/belt 600. The strap/belt 600 is configured to be placed on the recipient so that the strap/belt 600 applies enough pressure to the apparatus 400 to cause the pressure applicator portion 401 to apply pressure to the magnetic device implanted in the recipient to inhibit motion of the magnetic device during exposure of the magnetic device to an externally generated magnetic field.

The securing mechanism of FIGS. 6A-6B can also include a chin strap 602 that is configured to be wrapped under the chin of the recipient, as shown in FIG. 6B. The chin strap 602 attaches to the strap/belt 600 on either side of the head 500 using fasteners (e.g., Velcro). The chin strap 602 helps to inhibit movement of the strap/belt 600.

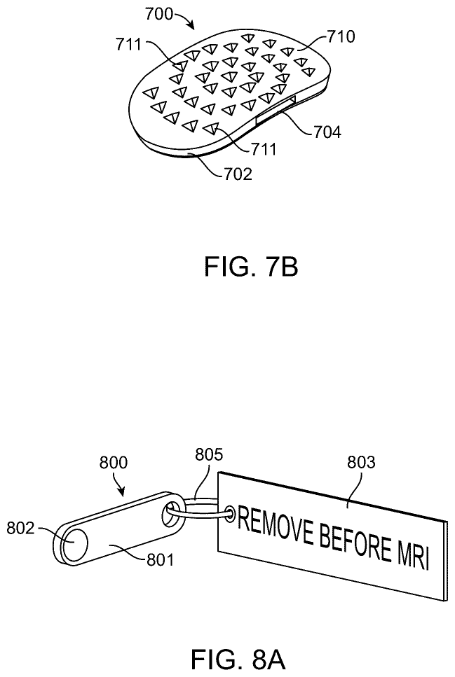

FIGS. 7A-7B are diagrams that depict another example of an apparatus 700 that is configured to be placed at a location over a magnetic device implanted in a recipient to inhibit motion of the magnetic device during exposure of the magnetic device to a magnetic field generated externally to the recipient. FIG. 7A illustrates a front view of the apparatus 700, and FIG. 7B illustrates a back view of the apparatus 700. FIG. 7A also shows a three dimensional x-y-z axis to assist in visualization of the apparatus 700. Apparatus 700 may, for example, be a splint. Referring to FIG. 7A, apparatus 700 includes a pressure applicator portion 701, a base portion 702, and a support portion 703. The pressure applicator portion 701 is a pressure applicator mound that is mounted on the base portion 702 and that extends from a front surface 705 of the base portion 702 in the z direction. The pressure applicator portion 701 is configured to apply a targeted pressure over the location of the magnetic device to inhibit motion of the magnetic device in the recipient in response to exposure to a magnetic field generated externally to the recipient, as with the pressure applicator portion 401 of apparatus 400.

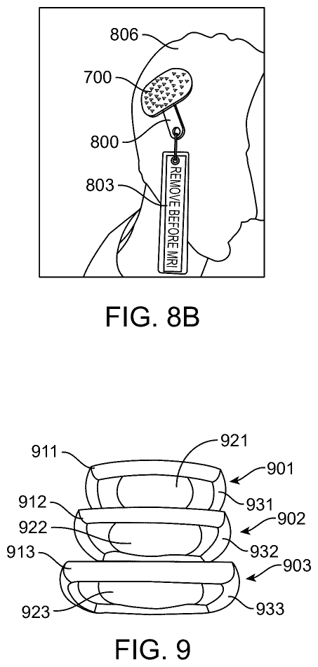

The base portion 702 is a curved backing plate having a rounded oval shape in the example of FIGS. 7A-7B. The base portion 702 can be flexible or rigid. The base portion 702 is curved along its length in the y direction. Base portion 702 includes a side cavity 704 that extends underneath the pressure applicator portion 701. Side cavity 704 is discussed in further detail below with respect to FIGS. 8A-8B. The support portion 703 can include stabilizing bumpers that are mounted on the base portion 702. The support portion 703 extends from the front surface 705 of the base portion 702 in the z direction around the perimeter of the base portion 702, as shown in FIG. 7A. The front surface 705 of the base portion 702 is recessed in the z direction relative to the pressure applicator portion 701 and relative to the support portion 703. The support portion 703 can have a curved upper surface that helps to reduce motion of apparatus 700 against the recipient. The pressure applicator portion 701, the base portion 702, and the support portion 703 of the apparatus 700 can be made of any suitable materials, including the exemplary materials discussed above with respect to apparatus 400. Referring to FIG. 7B, the base portion 702 also includes a back surface 710 that is textured with several pyramid-shaped spikes 711. The spikes 711 are configured to provide additional grip underneath the strap/belt 600 of FIGS. 6A-6B to inhibit movement of the apparatus 700 under the strap/belt 600.

FIGS. 8A-8B are diagrams that depict an example of an implant locator 800 for use with the apparatus 700 depicted in FIGS. 7A-7B. Implant locator 800 is configured to locate a magnetic device implanted in a recipient (e.g., internal magnet 233) to facilitate placement of the apparatus 700 over the magnetic device prior to exposing the recipient to an externally generated magnetic field. Referring to FIG. 8A, the implant locator 800 includes a rod 801 and a magnet 802 attached to one end of the rod 801. The rod 801 can be made of any suitable material, such as plastic. The end of the rod 801 that is attached to the magnet 802 is configured to fit inside the side cavity 704 of the apparatus 700 of FIGS. 7A-7B. The end of the rod 801 that is attached to the magnet 802 is inserted inside the side cavity 704 of the apparatus 700 prior to placement of the apparatus 700 on the recipient. When the end of the rod 801 attached to the magnet 802 is inside the side cavity 704 of the apparatus 700, the magnet 802 sits in the center of the apparatus 700, underneath the pressure applicator portion 701.

The apparatus 700 containing the implant locator 800 inserted partially into the side cavity 704 is then placed on the recipient over the location of the implanted magnetic device, as shown for example, in FIG. 8B. The magnet 802 has the opposite magnetic pole of the magnetic device implanted in the recipient. Because the magnet 802 and the implanted magnetic device have opposite magnetic poles, the magnet 802 is attracted to the implanted magnetic device so that the apparatus 700 containing the implant locator 800 is easily placed directly over the magnetic device implanted in the recipient. Because the magnet 802 sits underneath the pressure applicator portion 701, the pressure applicator portion 701 can easily be positioned directly over the magnetic device, while the apparatus 700 is placed on the recipient. The strap/belt 600 can then be placed on the recipient to apply sufficient pressure to the apparatus 700 to cause the pressure applicator portion 701 to inhibit movement of the magnetic device during exposure of the magnetic device to an externally generated magnetic field.

Thus, the implant locator 800 can be used to ensure that the pressure applicator portion 701 of the apparatus 700 is correctly positioned over the magnetic device prior to exposing the recipient to an externally generated magnetic field. During the application of the strap/belt 600 to the recipient, the implant locator 800 should remain in the apparatus 700 to prevent movement of the apparatus 700 and to maintain the correct positioning of the apparatus 700 on the recipient. The implant locator 800 can reduce the time and effort needed to place the apparatus 700 on the recipient directly over the magnetic device prior to exposing the recipient to an externally generated magnetic field. The implant locator 800 helps to ensure that the apparatus 700 is consistently placed over a magnetic device implanted in each recipient.

In some embodiments, the implant locator 800 can be attached to a label 803 via a ring 805 looped through a hole in rod 801 to ensure that the implant locator 800 is not left within the apparatus 700 during exposure to an externally generated magnetic field, as shown for example, in FIGS. 8A-8B. The label 803 can contain an easily visible reminder message, such as “Remove Before MRI.” In some embodiments, a head sock 806 can be placed over the head of the recipient to prevent direct contact between the apparatus 700 and the recipient, which reduces the need to clean the apparatus 700 between uses.

FIG. 9 is a diagram that depicts additional examples of three apparatuses 901, 902, and 903 that are each configured to be placed over a magnetic device implanted in a recipient so as to inhibit movement of the magnetic device during exposure of the magnetic device to an externally generated magnetic field. The apparatus 901 has a base portion 911, a pressure applicator portion 921, and a support portion 931. The apparatus 902 has a base portion 912, a pressure applicator portion 922, and a support portion 932. The apparatus 903 has a base portion 913, a pressure applicator portion 923, and a support portion 933. Each of the base portions 911-913 of the apparatuses 901-903, respectively, has a different curvature. The base portion 911 of the apparatus 901 has more curvature than the base portion 912 of apparatus 902. The base portion 913 of the apparatus 903 is flat with no curvature. The pressure applicator portions 921-923 of apparatuses 901-903, respectively, can be placed over magnetic devices implanted in recipients at different locations in the recipients having different curvatures to inhibit movements of the magnetic devices during exposures to externally generated magnetic fields. The support portions 931-933 support the apparatuses 901-903 against the recipients to inhibit movements of the apparatuses 901-903 when the pressure applicator portions 921-923, respectively, apply pressure over the magnetic devices.

Any embodiment or any feature disclosed herein can be combined with any one or more other embodiments and/or other features disclosed herein, unless explicitly indicated otherwise. Any embodiment or any feature disclosed herein can be explicitly excluded from use with any one or more other embodiments and/or other features disclosed herein, unless explicitly indicated otherwise. It is noted that any method detailed herein also corresponds to a disclosure of a device and/or system configured to execute one or more or all of the method actions associated with the device and/or system as detailed herein. It is further noted that any disclosure of a device and/or system detailed herein corresponds to a method of making and/or using that device and/or system, including a method of using that device according to the functionality detailed herein.

The foregoing description of the exemplary embodiments of the present invention has been presented for the purpose of illustration. The foregoing description is not intended to be exhaustive or to limit the present invention to the examples disclosed herein. In some instances, features of the present invention can be employed without a corresponding use of other features as set forth. Many modifications, substitutions, and variations are possible in light of the above teachings, without departing from the scope of the present invention.

1. An apparatus for inhibiting movement of a magnetic device relative to a recipient that is caused by exposing the magnetic device to a magnetic field generated externally to the recipient, wherein the magnetic device is implanted in the recipient, the apparatus comprising:
a pressure applicator portion configured to apply pressure against the recipient at a location over the magnetic device when the apparatus is pressed against the recipient; and
a support portion configured to support the apparatus against the recipient at a different location than the location over the magnetic device when the pressure applicator portion applies pressure at the location over the magnetic device, wherein the location and the different location are spaced apart from each other.

2. The apparatus of claim 1 further includes a base portion from which both the pressure applicator portion and the support portion extend.

3. The apparatus of claim 2, wherein the base portion includes a surface that extends between, and that is recessed relative to, the pressure applicator portion and the support portion.

4. The apparatus of claim 1, wherein the support portion substantially surrounds the pressure applicator portion.

5. The apparatus of claim 2, wherein the support portion extends substantially around a perimeter of the base portion.

6. The apparatus of claim 2, wherein one or more of the pressure applicator portion, the support portion, and the base portion is/are curved.

7. The apparatus of claim 1, wherein the apparatus further comprises a cavity adapted to removably receive a magnetic implant locator.

8. The apparatus of claim 1, wherein the pressure applicator portion comprises a mound.

9. The apparatus of claim 1, wherein the support portion comprises a flexible material.

10. A method for inhibiting motion of an implanted magnetic device in a recipient in response to a magnetic field that is generated externally to the recipient, wherein the method comprises:
placing an apparatus on the recipient such that a first portion of the apparatus contacts the recipient at a location that is over the implanted magnetic device, and such that a second portion of the apparatus contacts the recipient at a location adjacent to the implanted magnetic device; and
securing the placed apparatus to the recipient such that the first portion and the second portion are pressed against the recipient so as to inhibit movement of the implanted magnetic device while the implanted magnetic device is exposed to the magnetic field.

11. The method of claim 10, wherein securing the placed apparatus to the recipient further comprises strapping the apparatus to the recipient.

12. The method of claim 10 further comprising:
placing a magnetic implant locator in a cavity of the apparatus to assist in aligning the first portion of the apparatus over the implanted magnetic device when placing the apparatus on the recipient.

13. The method of claim 12 further comprising removing the magnetic implant locator from the cavity after securing the placed apparatus to the recipient.

14. A device for limiting motion of a magnetic implant in a recipient caused by an externally generated magnetic field, the device comprising:
a first portion that is able to apply pressure on the magnetic implant when the device is secured to the recipient; and
a second portion that is able to limit movement of the secured device relative to the recipient during exposure of the magnetic implant to the externally generated magnetic field.

15. The device of claim 14 further comprising a third portion from which the first portion and the second portion extend.

16. The device of claim 15, wherein the third portion comprises a curved surface.

17. The device of claim 15, wherein at least one of the first portion, the second portion, and the third portion comprises a flexible material.

18. The device of claim 14, wherein the device further comprises a textured surface adapted to inhibit relative movement between the device and a strap that is used to secure the device to the recipient.

19. The device of claim 18, wherein the textured surface includes a plurality of spikes.

20. The device of claim 14, wherein the second portion comprises one or more bumpers.
